(19) United States
(12) Patent Application Publication
(10) Pub. No.: US 2011/0224256 A1
(43) Pub. Date: Sep. 15, 2011

(54) THERAPEUTIC IMPRINTING FOR THE TREATMENT OF PSYCHIATRIC DISORDERS

(76) Inventor: Susan L. Andersen, Medfield, MA (US)

(21) Appl. No.: 12/996,875

(22) PCT Filed: Jun. 12, 2009
(86) PCT No.: PCT/US09/47158

§ 371 Date: May 27, 2011

Related U.S. Application Data
(60) Provisional application No. 61/061,090, filed on Jun. 12, 2008.

(51) Int. Cl. A61K 31/4458; C07C 211/42; A61K 31/135; C07D 207/09; A61K 31/40; C07D 211/34; A61P 25/00; A61P 25/30; A61P 25/24; A61P 25/18

(52) U.S. Cl. 514/317; 564/428; 514/657; 548/567; 514/428; 546/238

(57) ABSTRACT

The invention features methods, kits, and compositions for the treatment of psychiatric disorders using therapeutic imprinting in pre-pubescent subjects.

### BACKGROUND OF THE INVENTION

Brain development is a remarkable process. Progenitor cells are born, differentiate, and migrate to their final locations. Axons and dendrites branch and form important synaptic connections that set the stage for encoding information potentially for the rest of life. In the mammalian brain, synapses and receptors within most regions are overproduced and eliminated by as much as 50% during two phases of life: immediately before birth and during the transitions from childhood to adolescence and from adolescence to adulthood. This process results in different critical and sensitive periods of brain development. Since Hebb (Hebb, D. O., “The organization of behavior,” New York: Wiley (1949)) first postulated that the strengthening of synaptic elements occurs through functional validation, researchers have applied this approach to understanding the sculpting of the immature brain. In this manner, the brain becomes wired to match the needs of the environment. Extensions of this hypothesis posit that exposure to both positive and negative elements before adolescence can imprint on the final adult topography in a manner that differs from exposure to the same elements after adolescence.

There is a need for new therapies which can ameliorate the symptoms of and/or reduce the risk of developing psychiatric disorders.

### SUMMARY OF THE INVENTION

Applicants have discovered that psychotropic agents can be used to alter the developmental trajectory of a pre-pubescent brain in a subject. Using the methods of the invention, a subject predisposed a psychiatric disorder can be treated to ameliorate the symptoms of the disorder using psychotropic agents which, in an adult brain, would only worsen the underlying neurochemical or physiological imbalance causing in the disorder.

In a first aspect, the invention features a method of ameliorating a symptom of a psychiatric disorder in a subject by altering the developmental trajectory of a pre-pubescent brain in a subject predisposed to the disorder including the steps of: (a) determining whether a pre-pubescent subject is predisposed to a psychiatric disorder; and (b) if the subject is predisposed to the disorder, administering to the subject a psychotropic agent that produces a neurochemical effect characteristic of the disorder in an amount sufficient to imprint on the brain a functional change that ameliorates a symptom of the disorder.

In one embodiment of the above aspect, the disorder is not a substance abuse disorder.

In certain embodiments the disorder is schizophrenia and the psychotropic agent is a dopamine agonist; the disorder is depression and the psychotropic agent is a serotonin antagonist; the disorder is PTSD and the psychotropic agent is a serotonin antagonist; the disorder is Tourette's syndrome and the psychotropic agent is a dopamine agonist; the disorder is ADHD and the psychotropic agent is a dopamine agonist; or the disorder is obsessive compulsive disorder and the psychotropic agent is a serotonin agonist. The psychotropic agent used in the methods of the invention can be any psychotropic agent described herein.

The invention also features a method of ameliorating a symptom of an addiction in a subject by altering the developmental trajectory of a pre-pubescent brain in a subject predisposed to the addiction including the steps of: (a) determining whether a pre-pubescent subject is predisposed to addiction; and (b) if the subject is predisposed to the addiction, administering to the subject a D3 dopamine agonist in an amount sufficient to imprint on the brain a functional change that ameliorates a symptom of addiction. In certain embodiments, the addiction is a substance abuse disorder. The D3 dopamine agonist can be, without limitation, any D3 dopamine agonist described herein.

In an embodiment of the above aspects, the psychotropic agent is administered at a low dosage, at a high dosage, or at a moderate dosage.

In another embodiment of the above aspects, the subject is from 6 to 12 years old, 4 to 14 years old, 5 to 13 years old, 6 to 10 years old, 7 to 12 years old, 8 to 12 years old, 9 to 12 years old, 10 to 12 years old, or 7 to 11 years old.

In still another embodiment of the above aspects, the psychotropic agent is administered to the pre-pubescent subject for a period of 6 months to 36 months, 6 months to 30 months, 6 months to 24 months, 6 months to 18 months, 10 months to 36 months, 10 months to 30 months, 10 months to 24 months, 10 months to 18 months, or 12 months to 36 months.

In another embodiment of the above aspects, the psychotropic agent is administered to the pre-pubescent subject intermittently.

In yet another embodiment of the above aspects, a determination about whether a pre-pubescent subject is predisposed to a psychiatric disorder is made using a genetic test, a cognitive test, a behavioral test, or by taking a family history.

In a related aspect, the invention features a kit including (i) a psychotropic agent; and (ii) instructions for administering the psychotropic agent to a pre-pubescent subject to treat or ameliorate a symptom of a psychiatric disorder, wherein the psychotropic agent produces a neurochemical effect characteristic of the disorder.

For example, the invention includes a kit in which disorder is schizophrenia and the psychotropic agent is a dopamine agonist; the disorder is depression and the psychotropic agent is a serotonin antagonist; the disorder is PTSD and the psychotropic agent is a serotonin antagonist; the disorder is Tourette's syndrome and the psychotropic agent is a dopamine agonist; the disorder is ADHD and the psychotropic agent is a dopamine agonist; the disorder is obsessive compulsive disorder and the psychotropic agent is a serotonin agonist; and the disorder is addiction and the psychotropic agent is a D3 dopamine agonist.

In one embodiment, the kit further includes instructions for administering the psychotropic agent to the pre-pubescent subject for a period of 6 months to 36 months, 6 months to 30 months, 6 months to 24 months, 6 months to 18 months, 10 months to 36 months, 10 months to 30 months, 10 months to 24 months, 10 months to 18 months, or 12 months to 36 months.

In another embodiment, the kit further includes instructions to administer the psychotropic agent to the pre-pubescent subject intermittently.

The psychotropic agent used in the kits of the invention can be any psychotropic agent described herein.

As used herein, the term “treating” refers to administering a pharmaceutical composition for prophylactic purposes. To “prevent disease” refers to prophylactic treatment of a subject who is not yet ill, but who is susceptible to, or otherwise at risk of, a particular disease. Thus, in the claims and embodiments, treating is the administration to a subject for prophylactic purposes.

As used herein, the terms “an amount sufficient” and “sufficient amount” refer to the amount of a psychotropic agent that when given to a pre-pubescent subject therapeutically imprints on the brain of the subject a functional change. The sufficient amount used to practice the invention for prophylactic treatment of psychiatric disorders results in a functional change that ameliorates the symptoms of psychiatric disorder and can vary depending upon the manner of administration, the age, body weight, and general health of the subject. Ultimately, the attending physician will decide the appropriate amount and dosage regimen. Such amount is referred to as a “sufficient” amount.

As used herein, to “ameliorate a symptom” is to reduce the severity of at least one symptom associated with, or to reduce the likelihood of developing, a psychiatric disorder in a prepubescent subject predisposed thereto using the methods of the invention. A symptom is ameliorated in comparison to the severity of the symptoms, or risk of disease predicted, for subjects in the absence of undergoing the therapy of the invention.

As used herein, the term “neurochemical effect” refers to a physiological effect produced by a psychotropic agent and a physiological condition characterized by a psychiatric disorder (e.g., reduced activity at serotonin receptors in the brains of subjects suffering from depression and the reduction of serotonin activity caused by the administration of a serotonin antagonist to a subject). The methods and kits of the invention feature administering to a subject a psychotropic agent that produces a neurochemical effect characteristic of a psychiatric disorder. Such therapy, when given to a prepubescent subject, can be used to imprint on the brain of the subject a functional change that alters the developmental trajectory of the brain, and in doing so ameliorates the symptoms of the psychiatric disorder.

By a “low dosage” is meant at least 5% less, 10% less, 20% less, 30% less, 40% less, 50% less, 60% less, 70% less, or even 80% less than the lowest standard recommended dosage of a particular compound formulated for a given route of administration for treatment of any human disease or condition.

By a “high dosage” is meant at least 5% more, 10% more, 20% more, 30% more, 50% more, 90% more, 150% more, or even 200% more than the highest standard recommended dosage of a particular compound for treatment of any human disease or condition.

By “intermittent administration” or “administering intermittently” is meant a particular dosing regimen for a particular psychotropic agent which, based upon the PK profile of the drug, is characterized by intermittent periods during the therapy in which the levels of psychotropic agent fall to sub-therapeutic levels prior to the next dosing of psychotropic agent administered to the subject. For psychotropic agents having short absorption and elimination half-lives, such as methylphenidate, intermittent administration can be achieved by, for example, dosing once daily, once every other day, once every three days, or once weekly. For psychotropic agents having long absorption and elimination half-lives, such as fenfluramine (i.e., to achieve steady-state concentrations may require a week or more of dosing BID), intermittent administration can be achieved by, for example, alternating periods of “on” and “off” dosing (e.g., 2, 4, 6, or 8 weeks of daily dosing during the “on” period, followed by 2, 4, 6, or 8 weeks without medication during the “off” period).

By a “moderate dosage” is meant the dosage between the low dosage and the high dosage.

Other features and advantages of the invention will be apparent from the following detailed description, the drawings, and the claims.

### BRIEF DESCRIPTION OF THE DRAWINGS

FIG. 1. Regions of interest (ROI) for all comparisons. (Upper) Anatomic images with the ROI used for the analyses with the following definitions: Cing, cingulate; LFPCx, lateral frontoparietal cortex; MPFCx, medial prefrontal cortex; NAcc, nucleus accumbens; Striatum and Thalamus. (Middle) Representative activation maps following challenge with 2 mg/kg i.v. dose of methylphenidate (MPH) in adult rats that had been treated with saline vehicle (VEH) between P20 and P35. (Lower) Representative activation maps following challenge with 2 mg/kg i.v. dose of MPH in adult rats that had been treated with MPH between P20 and P35. Statistical tmaps of degree of change are on the right side.

FIG. 2. Graphic comparison of regional cerebral blood volume (rCBV) maps as shown in FIG. 1 of adult animals exposed to either methylphenidate (MPH; 2 mg/kg, twice daily) or vehicle (VEH) between P20 and P35 following MPH challenge. Means±SEM for n=7 rats are presented. *P<0.01, #P<0.08.

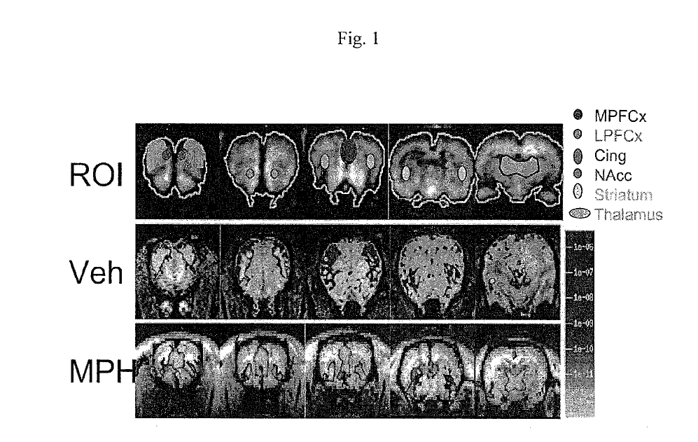

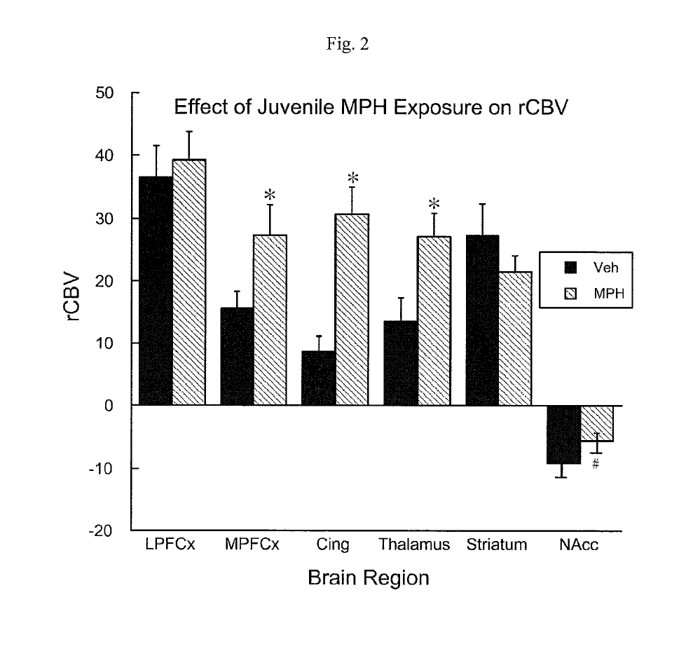

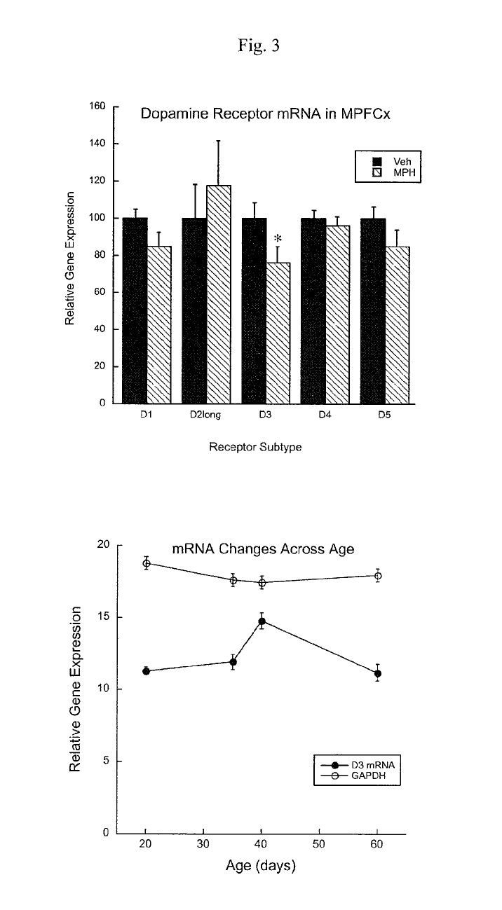

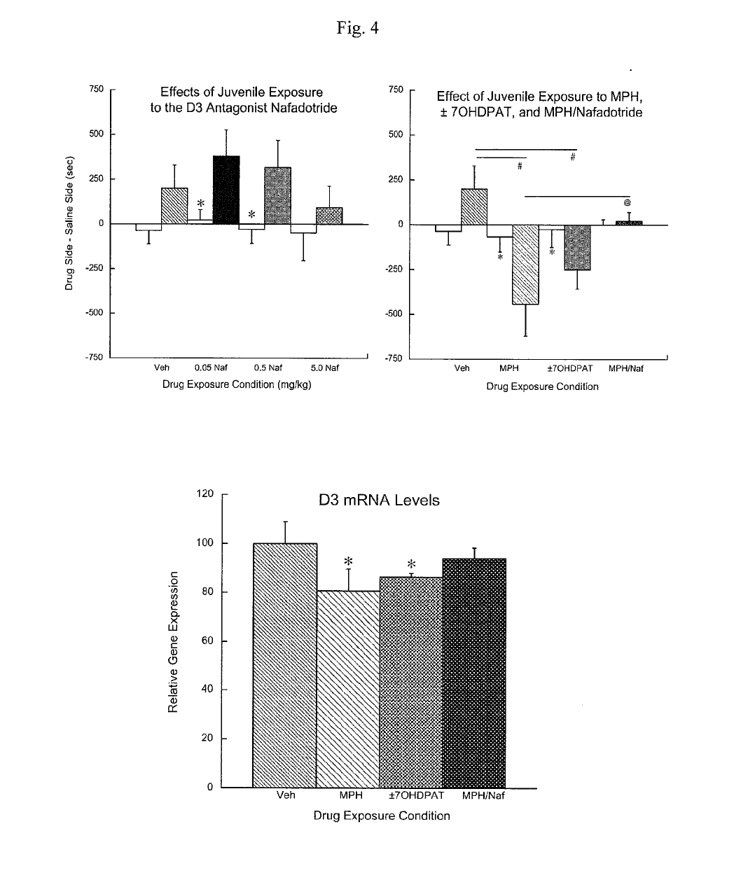

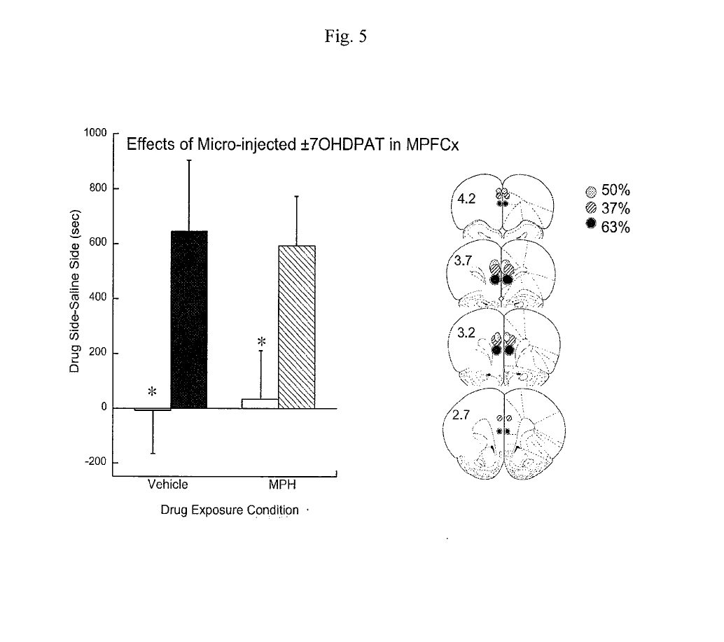

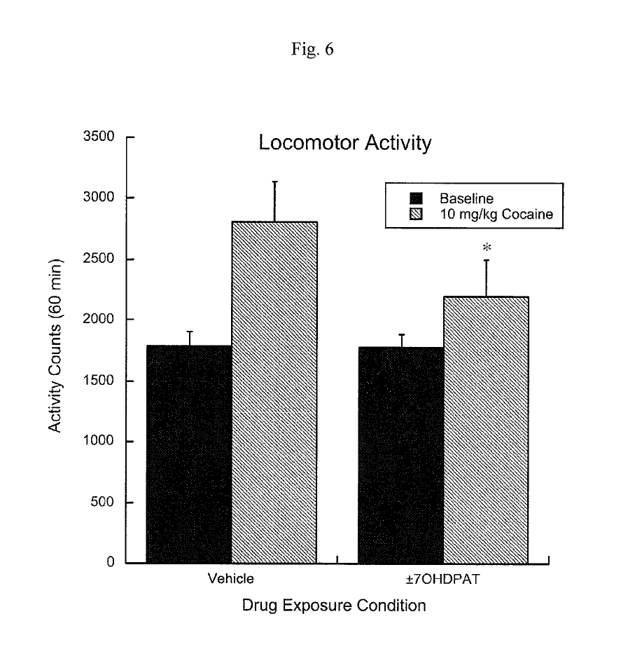

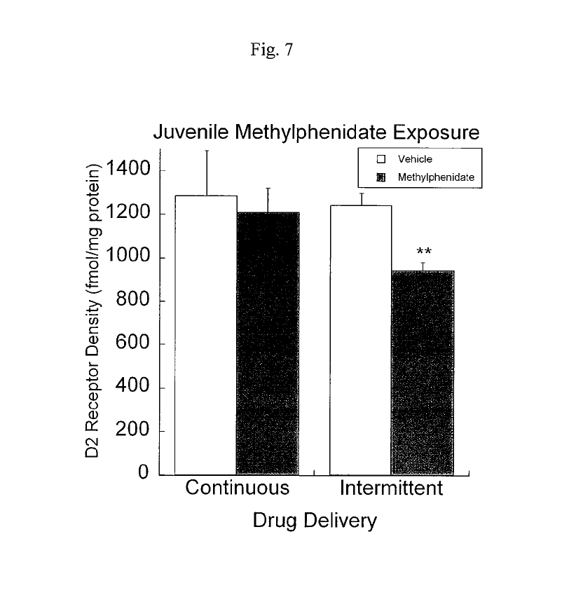

### Abbreviations as in FIG. 1.

FIG. 3. Dopamine D3 receptor-related changes in response to methylphenidate (MPH) and in normal development. (Upper) D1, D2 long, D3, D4 and D5 dopamine receptors mRNA levels in the medial prefrontal cortex (MPFCx) of MPH-treated rats 25 days after cessation of MPH. mRNA expression was determined by Q-PCR and data were analysed by the 2-ΔΔCt method of Livak & Schmittgen (Livak, K. J. & Schmittgen, T. D. Methods, 25:402-408 (2001)). Means±SE are expressed as a percentage of vehicle (VEH)-treated control subjects (n=9 subjects per condition). Each Q-PCR analysis was performed in triplicate and each value was corrected using its corresponding glyceraldehyde 3-phosphate dehydrogenase (GAPDH) expression level (*indicates significant t-test at P<0.05). (Lower) Age-related changes in D3 and GAPDH mRNA expression in the MPFCx in normal, untreated animals at P20, 35, 40 and 60. Means±SE of n=6 subjects.

FIG. 4. Behavioral effects of D3 manipulations. Data are expressed as time spent in the drug side minus time spent in the saline side before and after conditioning. Significant place conditioned effects are indicated by comparing pre-conditioned effects (open bars) vs. post-conditioning effects (patterned bars); *P<0.05. (Left) Dose-response of effects of juvenile nafadotride (Naf) treatment on place conditioning to 10 mg/kg cocaine environments. Juvenile exposure to 0.05 and 0.5 mg/kg Naf animals resulted in a significant preference for the cocaine-associated environment, whereas animals exposed to vehicle (VEH; P=0.11) or 5.0 mg/kg (P>0.7) did not form such a preference. Means±SEM for n=7-9 rats/group presented. (Right) Methylphenidate (MPH) and ±7-OHDPAT juvenile exposed animals had an aversion to the cocaine-associated environment relative to pre-conditioned behavior (*P<0.05), and significantly differed from VEH (P<0.05). Combined exposure to MPH and 0.05 mg/kg Naf reversed this aversion (@P<0.05). (Bottom right) D3 mRNA levels from the animals shown in top, right (n=8-9 rats/group). Data were corrected using individual GAPDH expression levels. (*indicates significant t-test at P<0.05).

FIG. 5. (Left) VEH and methylphenidate (MPH)-exposed subjects demonstrated a significant preference for cocaine-associated environments (main effect of conditioning: *P<0.05) following 10 lg/0.3 lL injection of ±7 OHDPAT into the medial prefrontal cortex (MPFCx) during conditioning. Means±SEM for n=5 rats/group presented. (Right) Location of cannula placement for microinjections as determined by Cresyl violet. The percentage of animals that was characterized by this placement is shown.

FIG. 6. Locomotor activity in response to a 10 mg/kg challenge with cocaine on the second day of cocaine conditioning in VEH- and ±7-OHDPAT-treated subjects (n=7-8 animals per group). *P<0.05 comparing the 10 mg/kg cocaine groups.

FIG. 7. In this study, rats were administered a dosage of 2 mg/kg methylphenidate either by osmotic minipump (continuous release) or twice daily by interperitoneal injection (intermittent) between the ages of postnatal day 20-35. D2 dopamine receptor binding was assessed in the striatum at 40 days of age using quantitative autoradiography (see Andersen et al., Synapse 37:167-169 (2000)). As shown in the FIG. 7, D2 receptor density was reduced significantly when given by an intermittent schedule (t9=4.53, P<0.005), but not when administered by continuous release (P>0.7).

### DETAILED DESCRIPTION

Applicants have discovered that psychotropic agents can be used to alter the developmental trajectory of a pre-pubescent brain in a subject. Using the methods and kits of the invention, a subject predisposed to a psychiatric disorder can be treated to ameliorate the effects a psychiatric disorder. For example, the symptoms of attention deficit hyperactivity disorder (ADHD), schizophrenia, and Tourette's syndrome can be ameliorated by administering a dopamine agonist; the symptoms of depression and post traumatic stress disorder (PTSD) can be ameliorated by administering a serotonin antagonist; and the symptoms of addiction can be ameliorated by administering a D3 dopamine agonist. The invention is described below in greater detail.

### Psychotropic Agents

Psychotropic agents which can be used in the methods and kits of the invention include, without limitation, dopamine agonists, serotonin antagonists, dopamine antagonists, and serotonin agonists.

The dopamine agonist for use in the treatment of ADHD, schizophrenia, Tourette's syndrome, and other psychiatric disorders associated with excess activity at dopamine receptors include, without limitation, dopamine precursors, such as L-dopa; dopaminergic agents, such as Levodopa-carbidopa (SINEMET®) or Levodopa-benserazide (PROLOPA®, MADOPAR®); and dopamine agonists, such as apomorphine, bromocriptine (PARLODEL®), cabergoline (DOSTINEX®), lisuride (DOPERGINE®), pergolide (PERMAX®), pramipexole (MIRAPEX®), and ropinirole (REQUIP®).

The D3 dopamine agonist for use in the treatment of addiction and other psychiatric disorders associated with excess activity at D3 dopamine receptors include, without limitation, 7-OH-DPAT, Pramipexole, Ropinirole, SB-277,011 (Stemp et al. J. Med. Chem. 45:1878 (2000)), R-(−)-3-(4-Propylmorpholin-2-yl)phenol hydrochloride, and S32504 (see U.S. Patent Publication No. 20060052435). Desirably, the D3 dopamine agonist for use in treating addiction has at least a 2-fold, 3-fold, 5-fold, or 10-fold selectivity for D3 receptors over D2 receptors.

The serotonin antagonist for use in the treatment of depression, post traumatic stress disorder, and other psychiatric disorders associated with reduced activity at serotonin receptors include, without limitation, ergonovine (ERGOTRATE®), ondansetron (ZOFRAN®), clozapine (CLOZARL®), risperidone (RISPERDAL®), methysergide (SANSERT®), cyproheptadine (PERIACTIN®), ritanserin, pizotifen, granisetron, metoclopramide, tropisetron, dolasetron, tri-methobenzamide, ketanserin, amitriptyline, azatadine, cyproheptadine, fenclonine, dexfenfluramnine, fenfluramine, chlorpromazine, and mianserin.

The serotonin agonist for use in the treatment of obsessive compulsive disorder and other psychiatric disorders associated with increased activity at serotonin receptors include, without limitation, almotriptan, bufotenin, buspirone, eletriptan, frovatriptan, gepirone, 8-hydroxy-2-(di-n-propylamino)tetralin (8-OH-DPAT), ipsapirone, naratriptan, quipazine, rizatriptan, sumatriptan, tegaserod, venlafaxine, and zolmitriptan.

### Formulation and Kits

Therapy according to the invention includes the administration of a psychotropic agent by any appropriate route for ameliorating a symptom of a psychiatric disorder. These may be administered to pre-pubescent humans along with a pharmaceutically acceptable diluent, carrier, or excipient, in unit dosage form. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, ear drops, or aerosols.

Administration of compounds in controlled release formulations is useful where the compound of formula I has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided in unit dosage form as chewable tablets, tablets, caplets, or capsules (i.e., as hard gelatin capsules wherein the active ingredient is mixed with an inert solid diluent, or as soft gelatin capsules wherein the active ingredient is mixed with water or an oil medium).

Therapy according to the invention may be performed at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of psychiatric disorder being treated, the age and condition of the patient, the stage and how the patient responds to the treatment.

The psychotropic agent for use in the therapies of the invention can be packaged as a kit. The kit can include optional components that aid in the administration of the unit dose to patients, such as vials for reconstituting powder forms, syringes for injection, customized IV delivery systems, inhalers, etc. Additionally, the unit dose kit can contain instructions for preparation and/or administration of the compositions. The kit may be manufactured as a single use unit dose for one patient, multiple uses for a particular patient; or the kit may contain multiple doses suitable for administration to multiple patients (“bulk packaging”). The kit components may be assembled in cartons, blister packs, bottles, tubes, and the like.

### ADHD and Addiction

Attention deficit hyperactivity disorder (ADHD) is a complex disorder with strong genetic and environmental influences that affects both motor and cognitive processes in approximately 3-6% of school-aged children (Barkley et al, J. Am. Acad. Child Adolesc. Psychiatry, 36:1204-1210 (1997); Teicher at al, in The American Psychiatric Press Textbook of Neuropsychiatry, American Psychiatric Press, Washington, D.C. (1997)). Pathophysiological evidence supports frontal-striatal-thalamic circuitry (Ernst et al., J. Neurosci., 18:5901-5907 (1998)) as a core neurobiological substrate of the primary symptoms of inattention, overactivity and reward deficiency (Ernst et al., Am. J. Psychiatry, 160:1061-1070 (2003); Cardinal & Everitt, Curr. Opin. Neurobiol., 14:156-162 (2004); Roth et al., Psychiatr. Clin. North Am., 27:83-96 (2004); Luman et al., Clin. Psychol. Rev., 25:183-213 (2005)). Cerebellar dysfunction also plays a role in the cognitive deficits associated with ADHD (Valera et al., Biol. Psychiatry, 57:439-447 (2005); Mackie et al., Am. J. Psychol., 164:647-655 (2007)). Genetic, neuroimaging and animal studies implicate the role of dopamine receptors D4 and D5 and the dopamine transporter, as well as changes within the noradrenergic system and the serotonin system (Thapar et al., Hum. Mol. Genet., 14:R275-R282 (2005); Faraone & Khan, J. Clin. Psychiatry, 67:13-20 (2006)). ADHD is effectively treated with psychostimulants, including methylphenidate (MPH; Ritalin™; Greenhill et al., J. Am. Acad. Child Adolesc. Psychiatry, 41:26S-49S (2002)), which are believed to ameliorate the deficits in reward processing when given acutely (Luman et al., Clin. Psychol. Rev., 25:183-213 (2005)).

Central to the processing of reward is the frontal cortex and the nucleus accumbens (London et al., Cereb. Cortex, 10:334-342 (2000); Elliott et al., J. Neurosci., 23:303-307 (2003); Knutson et al., Neuroimage, 18:263-272 (2003); Ernst et al., Neuropsychologia, 42:1585-1597 (2004); Schulz et al., Am. J. Psychiatry, 161:1650-1657 (2004)). Data from imaging (Ernst et al., J. Neurosci., 18:5901-5907 (1998); Schweitzer et al., Biol. Psychiatry, 56:597-606 (2004); Volkow et al., Am. J. Psychiatry, 161:1173-1180 (2004)) and behavioral studies (Cardinal et al., Curr. Opin. Neurobiol., 14:156-162 (2004) and Ann. N. Y. Acad. Sci., 1021:33-50 (2004)) implicate these brain areas, and suggest that they are common to both the pathophysiology of ADHD and addiction (Schulz et al., Am. J. Psychiatry, 161:1650-1657 (2004)). MPH increases cerebral blood metaboslism and flow (Vaidya et al., Proc. Natl Acad. Sci. U.S.A., 95:14494-14499 (1998); Kim et al., Yonsei Med. J., 42:19-29 (2001)), and this in turn is posited to improve reward processing.

Deficits in reward processing may explain a high rate of substance abuse among individuals with ADHD (Saules et al., J. Addict. Dis., 22:71-78 (2003)). While somewhat controversial, childhood treatment of ADHD with stimulants does not increase (Loney et al., Attention Deficit Hyperactivity Disorder-State of the Science-Best Practices (Jensen, P. S. & Cooper, J. R., Eds) Civic Research Institute, Kingston, N.J., pp. 1-16, (2002)) and may decrease (e.g. Biederman et al., Biol. Psychiatry, 60:1111-1120 (1999)), risk for substance use disorders later in life relative to untreated cohorts. Parallel findings of reduced risk following stimulant exposure are modeled in animals. For example, rats treated as juveniles have reduced preference for cocaine associated environments and demonstrate decreased cocaine-induced locomotion in adulthood (Dow-Edwards et al, Pharmacol. Biochem. Behay., 70:23-30 (2001); Andersen et al., Nat. Neurosci., 5:13-14 (2002)). These behavioral effects, however, depend on the timing of exposure, as adolescent or adult exposure produces the opposite effects (Brandon et al., Neuropsychopharmacology, 25:651-661 (2001); Andersen et al., Nat. Neurosci., 5:13-14 (2002); Guerriero et al., Biol. Psychiatry, 1171-1180 (2006)). Together, preclinical and clinical (Castellanos et al., JAMA, 288:1740-1748 (2002); Hyman, Biol. Psychiatry, 54:1310-1311 (2003)) studies suggest long-term effects of juvenile stimulant exposure on brain maturation (Andersen, Trends Pharmacol. Sci., 26:237-243 (2005)).

Similar behavioral findings between humans and animals imply a common mechanism of action of enduring drug exposure. Neuroimaging provides a translational opportunity to characterize changes in blood flow that can be further dissected mechanistically in animals. Cortical changes in regional cerebral blood volume (rCBV) were examined with magnetic resonance imaging (MRI) in response to drug challenge in adulthood. Because MPH increases extracellular levels of dopamine in the medial prefrontal cortex (MPFCx; Berridge et al., Biol. Psychiatry, 60:1111-1120 (2006)), we assessed whether dopamine receptors were changed following juvenile MPH exposure. Previous research suggests that a prenatal sensitive period exists for the programming of D3 receptors by cocaine (Silvers et al., Neurotoxicol. Teratol., 28:173-180 (2006)). However, given the postnatal overproduction of the cortical D1 and D2 family of dopamine receptors (Andersen et al., Synapse, 37:167-169 (2000); Tarazi et al., Int. J. Dev. Neurosci., 18:29-37 (2000)), a second sensitive period may exist.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Abbreviations

ADHD, attention deficit hyperactivity disorder;

BDNF, brain-derived neurotrophic factor;

Cing, cingulate;

GAPDH, glyceraldehyde 3-phosphate dehydrogenase;

LFPCx, lateral frontoparietal cortex;

MION, microcrystalline iron oxide nanocolloid;

MPFCx, medial prefrontal cortex;

MPH, methylphenidate;

MRI, magnetic resonance imaging;

Naf, nafadotride;

±7-OHDPAT, ±7-hydroxy-N,N-di-n-propyl-2-aminotetralin;

Q-PCR, quantitative polymerase chain reaction;

rCBV, regional cerebral blood volume;

ROI, regions of interest;

Thal, thalamus;

VEH, vehicle.

### Materials and Methods

Subjects

Lactating female Sprague-Dawley rats obtained from Charles River (Worcester, Mass., USA) were housed with their litters on a 12:12 h light:dark cycle, with lights on at 07.00 h with food and water provided ad libitum. Litters were culled to eight pups of equal numbers of males and females on postnatal day 1 (P1) and weaned at P21. Only males were used for these studies, with only one subject/litter in any individual condition, and the remaining littermates used in other studies. Four groups of untreated subjects at P20, 35, 40 and 60 were killed to determine normal development, whereas the remaining groups were treated as described below. All animals were treated in accordance with the policies established by NIH and the McLean Hospital Institutional Animal Care and Use Committee.

Drugs

d,l-Methylphenidate HCl (MPH), ±7-hydroxy-N,N-di-n-propyl-2-aminotetralin (±7-OHDPAT) and cocaine HCl were obtained from Sigma (St Louis, Mo., USA). Nafadotride (Naf) was obtained from Tocris (Ellisville, Mo., USA). Each drug was dissolved in 0.9% saline (vehicle, VEH) and administered in a volume of 1 mL/kg by Hamilton syringe. Doses are based on the salt form of each drug.

Drug Treatment

Juvenile Sprague-Dawley male rats were treated with MPH (2 mg/kg, i.p. twice daily), ±7-OHDPAT (0.3 mg/kg, i.p. twice daily), Naf (0.05, 0.5 and 5 mg/kg; i.p. twice daily) or VEH (i.p. twice daily) between the ages of P20 and P35 (Andersen et al., Nat. Neurosci., 5:13-14 (2002)). The MPH dose was selected on the basis of previous studies (see Andersen et al., Nat. Neurosci., 5:13-14 (2002); Bolanos et al., Biol. Psychiatry, 54:1317-1329 (2003); and Mague et al., Biol. Psychiatry, 57:120-125 (2005)), unpublished observations that we find similar behavioral effects if MPH is given i.p. or orally at this dose, and evidence that it approximates a clinically relevant dose in humans based on plasma levels (see Wargin et al., J. Pharmacol. Exp. Ther., 226:382-386 (1983); and Kuczenski, R. & Segal, D. S. J. Neurosci., 22:7264-7271 (2002)). Clinically therapeutic doses of MPH yield plasma levels between 8 and 40 ng/mL (Swanson et al., Behay. Brain Res., 130:73-78 (2002); Teicher et al., Ann. N. Y. Acad. Sci., 1071:313-323 (2006)), with 0.5 mg/kg i.p., achieving plasma levels of 36 ng/mL within 5 min in rats (Berridge et al., Biol. Psychiatry, 60:1111-1120 (2006)). Doses for juvenile exposure to ±7-OHDPAT are based on Neisewander et al., Neuropsychopharmacology, 29:1479-1487 (2004), and cortical microinjections of 10 lg/side were derived from Boulay et al. Prog. Neuropsychopharmacol. Biol. Psychiatry, 24:39-49 (2000). Naf doses below 1 mg/kg have negligible D2 receptor occupancy (Sautel et al., J. Pharmacol. Exp. Ther., 275:1239-1246 (1995)) and are behaviorally effective (Richtand et al., Brain Res., 867:239-242 (2000)). Subjects were weighed daily at 09.00 h and injected with their assigned drug; the second injection was administered 4 h later at 13.00 h. This regimen is designed to match the twice-daily dosing used by children, taking into account the higher metabolic rates of rats (Wargin et al., J. Pharmacol. Exp. Ther., 226:382-386 (1983)). After the second drug administration on P35, the rats received no further treatments until behavioral testing at P60.

MRI Methods

MRI was conducted in two groups of animals (MPH-treated or VEH treated; n=7) at approximately 60 days old. Functional changes in rCBV were determined by drug-induced alterations following paramagnetic contrast agent microcrystalline iron oxide nanocolloid (MION) in anesthetized treated subjects (Chen et al., 2001). All experiments were performed at 2 T or 4.7 T on a GE Omega CSI instrument (GE, Fremont, Calif., USA) using a home-built surface coil for transmit-receive and immobilization of the head using a rigid plastic head holder. All VEH/MPH subjects were matched for scanner strength to control for any possible differences due to magnet strength (Mandeville et al., Magn. Reson. Med., 52:1272-1281 (2004)). All animals received 1% halothane/N₂O/O₂ anesthesia during the imaging procedure. Animals were kept at 37° C. using a circulating water blanket heater. Tail veins were accessed for administration of contrast agent, drug challenge, arterial blood gas sampling and on-line blood pressure monitoring, as described in Chen et al. (Neuroreport, 10:2881-2886 (1999)) and Mandeville et al., Magn. Reson. Med., 45:443-447 (2001).

rCBV with MION Contrast

According to standard methods, 20 baseline images were collected, followed by a 10 mg/kg injection of MION (an iron particle-based contrast agent that produces long-lasting intravascular contrast for T2* contrast measures) and an additional 20 baseline images. Images were collected for 1-2 h after an i.v. injection with MPH (2 mg/kg) to characterize drug-induced changes, which optimally brackets MPH drug effects. Typical parameters are TR/FE 1000/20 ms (at 4.7 T) with a 25 mm FOV; eight 1.0-mm slices and a 128×64 matrix. Images were measured serially with a temporal resolution of 2 min.

Anatomic Registration of MRI Functional Images

All functional images within each condition were registered onto the same standard brain template to facilitate comparisons between the experimental groups. Registration parameters were determined for each functional scan using a single full-brain image set that is the average of all baseline time points prior to drug injection. These registration parameters were applied to all time points in the functional data set. This approach is valid, as the animals are of the same size, experimental treatment and imaging protocol, and are anesthetized, which eliminates motion artifacts.

phMRI Image Analysis

The images were processed as described in Chen et al. (Neuroreport, 10:2881-2886 (1999)) and Mandeville et al. (Magn. Reson. Med., 45:443-447 (2001)). Briefly, topographic maps (t-maps) were calculated using the pre-MPH post-MION baselines vs. the post-MPH time points. Additionally, maps of rCBV were made using the relationship that rCBV=−ln(S/S0)/TE on a pixel-by-pixel basis. rCBV changes at the maximal effect are presented here. The spatial distribution of signal changes was converted to a statistical map of P-values using a t-test, comparing on a pixel-by-pixel basis the baseline vs. activated signal changes within a given region of interest (ROI). In cases where the signal returns to baseline, the data were corrected by a linear drift term. CBV was determined from the images by constructing pixel-by-pixel maps of the DR2* changes. Image intensities were converted to temporal changes in percent CBV as [(V(t)/V(0))=v(R2*(t))/(R2*(0))1)], where the change in transverse relaxation rate is calculated from the gradient echo time (TE), the average value of signal prior to injection of contrast agent (S0), and the signal during the functional run (S(t)) after injection of contrast agent: R2*(t)=−ln{S(t)/S0}/TE. The baseline value of this relaxation rate was determined from the average value of S(t) before injection of drug (SBASE): R2*(t)=)ln (Wallace et al., Synapse, 23:152-163. (1996))/TE using software from Massachusetts General Hospital. Linear drift corrections for the change in MION concentrations (which are minimal) over the time course of the experiment were performed. Measures of the rCBV change following peak drug challenge were assessed relative to individual baselines.

D3 Receptor Analysis

Animals were killed by decapitation. Three sets of subjects were used in the biochemical analyses. A first set of rats was treated only with MPH or VEH (n=6-8), but remained naïve to cocaine exposure at P60 (MPH-naive and VEH-naive). This group of rats was used to assess changes in dopamine receptor levels independent of any secondary effects of cocaine conditioning. The second set received either MPH or VEH (n=6-8), and underwent place conditioning to cocaine at P60 (see below), were killed and the MPFCx assessed for D3 receptor levels. A third set of subjects was unadulterated and killed at one of four ages (P25, P35, P40 and P60; n=6/age) for analysis of D3 levels over normal development.

Quantitative, Real Time-Polymerase Chain Reaction (Q-PCR)

For mRNA preparation, brains were rapidly dissected into the three main dopaminergic ascending projection targets: the PFC, nucleus accumbens and striatum. Sections were snap-frozen in TriReagent solution (Sigma) and stored at −80° C. until further analysis. Samples were homogenized, and total mRNA was prepared and quantified as described (Sonntag et al., Mol. Cell. Neurosci., 28:417-429 (2005)). Briefly, mRNA was DNAse digested (Ambion, Austin, Tex., USA) prior to reverse transcription into cDNA using the SuperScript II First-Strand Synthesis System (Invitrogen, Carlsbad, Calif., USA). cDNA samples were diluted to 50 ng/1 L mRNA equivalent. Between 2 and 4 μL of cDNA was analysed with Q-PCR using the SYBR Green Jumpstart Taq ReadyMix (Sigma). Amplifications were performed in a total volume of 25 μL with 40 nMol primers for each reaction (Opticon MJ thermocycler; MJ Research, Watertown, Mass., USA). Primer sequences were based on published methods when available, or designed according to known gene sequences (Table 1). PCR products were confirmed by restriction digest and revealed homogeneous melting curves in the Q-PCR assays. The expression of dopamine receptors was normalized to the housekeeping gene product glyceraldehyde 3-phosphate dehydrogenase (GAPDH). Linearity (r=0.9 or better) and detection limit of the assay were determined in successive 10-fold serial dilutions, performed in triplicate, and an optimal amount of template was chosen for the quantitative analysis.

Quantification was performed at a threshold detection line (‘threshold cycles’, Ct value). The Ct of each gene was normalized against GAPDH, which was run simultaneously for each marker. The ΔΔCt for each candidate was calculated as ΔCt of [Ct (candidate)−Ct (GAPDH)], according to the methods of Livak et al., Methods, 25:402-408 (2001), and plotted as percent change relative to VEH (Bahi et al., Eur. J. Neurosci., 21:3415-3426 (2005)). Table 1 is a list of primers used to characterize dopamine receptors and GAPDH.

TABLE 1

| Receptor | Primer | Reference |
|---|---|---|
| D1-F | 5′-AGATGACCCCCAAAGCAG-3′ | a |
| Dl-R | 5′-ACGTCCTGCTCAACCTTG-3′ | a |
| D2-F | 5′-CAGACCATGCCCAATGGC-3′ | a |
| D2-R | 5′-CACACCGAGAACAATGGC-3′ | a |
| D3-F | 5′-AAGCGCTACTACAGCATCTGC-3′ | b |
| D3-R | 5′-GGATAACCTGCCGTTGCTGAG-3′ | c |
| D4-F | 5′-CCTGATGTGTTGGGACGCCTTTC-3′ | c |
| D4-R | 5′-TGGTGTAGATGATGGGGTTGAGGG-3′ | c |
| D5-F | 5′-AAAGACTGGCTTCCCTTGTGTC-3′ | c |
| D5-R | 5′-CTGATGTTTACCGTCTGCACTG-3′ | c |
| GAPDH-F | 5′-AACTCCCATTCTTCCACCTTTG-3′ | c |
| GAPDH-R | 5′-CCCTGTTGCTGTAGCCATATTC-3′ | c |

a. Gross et al., Brain Res. Mol. Brain Res., 60: 1171-1180 (2000).
b. Flores et al., Neuroscience 91: 549-556 (1999).
c. Brenhouse, H.C., Sonntag, K.C. & Andersen, S.L. J. Neurosci., 28: 2375-2382 (2008).

Place Conditioning

Rats treated between P20 and P35 with either Naf (0, 0.05, 0.5 or 5.0 mg/kg; n=7-9), ±7-OHDPAT (0.3 mg/kg; n=9), MPH (2 mg/kg; n=9), or a combination of 2 mg/kg MPH and 0.05 mg/kg Naf (MPH/Naf; n=8) were tested at P60 for place conditioning to 10 mg/kg cocaine. According to standard laboratory methods (Andersen et al., Nat. Neurosci., 5:13-14 (2002); Carlezon, Methods Mol. Med., 84:243-249 (2003)), unbiased place conditioning chambers consisted of two large (24×18×33) side compartments separated by a small (12×18×33 cm) middle compartment. Screening was conducted for 30 min on Day 1. Rats were placed in the middle compartment and allowed to freely explore the apparatus. Rats that demonstrated a clear preference for either side (>18 of 30 min) were eliminated from further testing (n=4 in all). Two days of conditioning (with two sessions per day) occurred on Days 2 and 3 for 60 min (Andersen et al., Nat. Neurosci., 5:13-14 (2002); Carlezon, Methods Mol. Med., 84:243-249 (2003)). During the conditioning sessions, rats received a 1 mL/kg i.p. injection of VEH in the morning (09.00 h) and confined to one side. The rats were then returned to the home cage. Four hours later, rats received 10 mg/kg cocaine and confined to the other side. Sides differed in floor texture, wall colors and lighting, and assignments were counterbalanced. On Day 4, rats were permitted to freely explore the entire apparatus for 30 min in a drug-free state.

Dopamine Receptor Expression in Response to MPH Treatment

mRNA expression levels were measured to determine the effect of juvenile MPH exposure on dopamine receptors. Changes in dopamine receptors D1, D2 long, D3, D4 and D5 were examined with Q-PCR, as specific ligands are not available for all of the receptor subtypes (FIG. 3, upper). In the MPFCx, a significant 23.8±6.7% reduction in D3 receptor mRNAwas observed in MPH-exposed subjects relative to VEH controls 25 days post-exposure (t16=3.153, P<0.001). This difference was slightly greater (a 28.9±8.3% reduction) when assessed at P40 (not shown). No other significant changes were found in any dopamine receptor subtypes in this region (P>0.1). In addition, D3 mRNA did not change in either accumbens or striatum (P>0.5; not shown). These results point to an important role that D3 may play in mediating the enduring changes following chronic MPH exposure in MPFCx.

D3 mRNA Receptor Development

In order to better understand how the timing of MPH exposure interacted with normal D3 receptor development in the MPFCx, we examined mRNA expression levels at P20 and 35 (the exposure interval), 40 and 60 when the majority of these assessments occurred. As shown in FIG. 3 (lower), D3 mRNA expression changed with age (F3,20=11.15, P<0.0001) and was stable during the injection interval of P20 and P35. It then increased its expression by 31.0±5.0% at P40, and then rapidly returned to pre-pubertal values by P60. The overexpression of the D3 receptor is consistent with previous observations of age-related changes in D1 and D2 receptors in the MPFCx at P40 (Andersen et al., Synapse, 37:167-169 (2000)). GAPDH mRNA did not change across age (P>0.1).

Behavior and Manipulations of the D3 Receptor

To further investigate the functional role of the D3 receptor in juvenile psychostimulant treatment, animals were pre-pubertally treated with the D3-preferring agonist ±7-OHDPAT, the D3 antagonist Naf or a MPH/Naf combination. First, an overall significant effect of place conditioning to cocaine-associated environments was observed (conditioning [pre- vs. post-]: F1,29=9.63, P<0.005). Individual comparisons of pre- vs. post-conditioning within exposure condition demonstrated significant preferences to cocaine-associated environments following juvenile exposure to the 0.05 and 0.5 mg/kg Naf doses, but not VEH and the 5.0 mg/kg dose (FIG. 4, left). Next, juvenile manipulations of the D3 receptor on place conditioning were assessed and compared with each other. Overall, there was an exposure•conditioning [pre- vs. post-]) interaction (F3,32=6.22, P<0.01). Place aversion was observed to the 10 mg/kg cocaine associated environment in MPH (t7=2.97, P<0.05) and ±7-OHDPAT (t9=2.44, P<0.05) subjects, but not in the MPH/Naf group (P>0.7), using the low doses of Naf that alone were not effective (FIG. 4, left). Post hoc comparisons showed significant differences between the conditioned effects of VEH relative to the MPH and ±7-OHDPAT group and the MPH vs. MPH/Naf groups (P<0.05). FIG. 4 (bottom, right) confirms that D3 mRNA significantly changed as a function of juvenile MPH exposure, and is reversed by simultaneous treatment with Naf (F3,20=8.76, P<0.001). Moreover, microinjections of ±7-OHDPAT (10 lg/side) into the MPFCx showed a significant overall effect of conditioning (F1,8=17.55, P<0.01; FIG. 5), thus reversing the aversion to cocaine demonstrated by the MPH subjects (FIG. 4, right). Finally, baseline locomotor activity in the ±7-OHDPAT juvenile-exposed group did not differ from VEH control animals at 60 days old (F1,16=0.003, P>0.9; FIG. 6). However, locomotor response to the second day of 10 mg/kg cocaine challenge in ±7-OHDPAT-exposed subjects was significantly reduced relative to VEH controls (F1,16=5.20, P<0.05; FIG. 6), consistent with previous reports of reduced cocaine-induced activity following juvenile MPH exposure (Andersen et al., Nat. Neurosci., 5:13-14 (2002)).

Discussion

We observed a dramatic, persistent change in the brain responses of animals exposed to chronic MPH over a period of time preceding puberty. These changes are mediated, in part, through changes in cortical D3 dopamine receptors. Relative to VEH-treated subjects, MPH challenge in adulthood significantly increased rCBV in the MPFCx in subjects exposed as juveniles to MPH. Furthermore, juvenile exposure to MPH decreased D3 receptor mRNA in the MPFCx of adult rats, compared with VEH-treated rats. MPH exposure occurred during a period immediately prior to an overproduction of D3 mRNA in this region, when a sensitive period for these effects may end. The relationship of these changes to drug-seeking behavior was also investigated with place conditioning (Cabib et al., Psychopharmacology (Berl), 125:92-96 (1996)). Here, juvenile exposure to both MPH and ±7-OHDPAT reduced responsiveness to cocaine-associated environments in adulthood. These enduring behavioral effects of juvenile MPH exposure were partially blocked by co-administration of a D3 antagonist. Finally, the behavioral effect was localized to the MPFCx with microinjections of ±7-OHDPAT, which produced a place preference to cocaine in MPH-exposed subjects. Taken together, juvenile exposure to MPH appears to increase stimulant-induced changes in cortical rCBV and decrease responsiveness to cocaine-associated cues in adulthood through a reduction of D3 receptors.

These findings demonstrate that chronic, juvenile exposure to psychostimulants produces regional and persistent changes in rCBV that have relevance to clinical observations (Vaidya et al., Proc. Natl Acad. Sci. U.S.A., 95:14494-14499 (1998); Kim et al., Yonsei Med. J., 42:19-29 (2001)). The exacerbated increase in MPFCx and Cing activation and associated thalamic circuitry following juvenile MPH may reflect the underlying pathophysiology of hypoactivity in frontal regions of patients with ADHD (Ernst et al., J. Neuropsychiatry Clin. Neurosci., 10:168-177 (1998)). Increased cortical activation following MPH challenge in ADHD individuals has been observed (Vaidya et al., Proc. Natl Acad. Sci. U.S.A., 95:14494-14499 (1998); Kim et al., Yonsei Med. J., 42:19-29 (2001)) in both naïve and medication exposed children. We believe that these effects may be permanently enhanced by medication. Conversely, no significant enduring effects of juvenile exposure were observed in the dorsolateral PFCx or the caudate putamen. In both humans (Teicher et al., Nat. Med., 6, 470-473 (2000)) and animals (shown here), acute exposure to stimulants increases blood flow in the striatum with no enhancement following chronic exposure. Thus, reduction in hyperactivity, which correlates with striatal measures of blood flow; (Teicher et al., Nat. Med., 6, 470-473 (2000)) may be more dependent on acute levels of drug.

From a mechanistic perspective, data from phMRI, Q-PCR and behavioral analyses suggest that pre-pubertal stimulant exposure works in part by reducing cortical D3 receptors later in life. D3 mRNA is normally overexpressed during a period when this receptor loses its autoinhibitory function in the MPFCx (Andersen et al., Naunyn Schmiedebergs Arch. Pharmacol., 356:173-181 (1997)). D3 receptors have been localized to both dopamine and non-dopamine neurons (Stanwood et al., J. Pharmacol. Exp. Ther., 295:1223-1231 (2000)). At this stage, it is unclear which population of neurons in the MPFCx overexpresses D3 mRNA, although this is currently under investigation by our laboratory.

In a recent review, Le Foll et al. (Neuropharmacology, 49:525-541 (2005)) dissect the relationship between D3 receptors and drug dependence in adulthood. They concluded that the D3 receptor in the nucleus accumbens is involved in modulating motivation and drug-conditioned stimuli (Di Ciano et al., Neuropsychopharmacology, 28:329-338 (2003); Sokoloff et al., CNS Neurol. Disord. Drug Targets, 5:25-43 (2006)), including the role of environmental cues on drug-seeking behavior (e.g. place preference). For example, locomotor sensitization to cocaine is related to an increase in postsynaptic D3 receptor density in the nucleus accumbens (Wallace et al., Synapse, 23:152-163 (1996)). D3 receptors are also localized to tyrosine-immunoreactive terminals, indicative of autoreceptor activity within the accumbens (Diaz et al., J. Neurosci., 20:8677-8684 (2000)). Exposure to cocaine also works by desensitizing this autoreceptor (Richtand et al., Neuropsychopharmacology, 28:1422-1432 (2003)). Together, a loss of inhibition through desensitization and/or receptor loss enhance the rewarding effects of cocaine as mediated by the accumbens.

From a developmental perspective, however, a sensitive period for autoreceptor changes within the MPFCx may exist pre-pubertally. Changes in D3 receptor expression (this study) and function (Andersen et al., Naunyn Schmiedebergs Arch. Pharmacol., 356:173-181 (1997)), along with other dopaminergic changes (Tseng et al., Cereb. Cortex, 15:49-57 (2005); Brenhouse et al., J. Neurosci., 28:2375-2382 (2008)), occur within the MPFCx during adolescence in rats. Drug exposure prior to these rearrangements may alter this transition within the MPFCx (Andersen, Trends Pharmacol. Sci., 26:237-243 (2005)). There is evidence of pre-pubertal autoreceptor-like activity of the D3 receptor in the cortex (Andersen et al., Naunyn Schmiedebergs Arch. Pharmacol., 356:173-181 (1997)) that wanes and is absent by adulthood (Booth et al., Brain Res., 662:283-288 (1994)). Juvenile MPH exposure may work in a similar process as described above in adults by reducing an autoregulatory mechanism that would allow dopamine levels to rise in the MPFCx. Recently, Jezierski et al. (J. Neurochem., 103:2234-2244 (2007)) showed that exposure to MPH during postnatal development produced increased dopamine release in the MPFCx. Moreover, decreased D3 receptor-mediated autoregulation is consonant with previous observations that D3 receptor antagonists enhance stimulant-induced changes in dopamine release and rCBV in adult rats (Chen et al., Psychopharmacology (Berl), 180:705-715 (2004); Schwarz et al., Synapse, 54:1-10 (2004)). Increased extracellular dopamine levels subsequently would increase activity at other dopamine receptors, including the D1 receptor that has been implicated in drug-cue associations (Kalivas, Neuron, 45:647-650 (2005); Brenhouse et al., J. Neurosci., 28:2375-2382 (2008)). Alternatively, juvenile MPH may work by decreasing the development of overproduced postsynaptic D3 receptors (FIG. 3; Stanwood et al., Neurosci. Lett., 223:13-16 (1997)). The localization of these receptors following MPH still remains to be elucidated. However, the effectiveness of D3 antagonists to block cue-related drug-seeking (Xi et al., Eur. J. Neurosci., 21:3427-3438 (2005); Xi et al, CNS Drug Rev., 13:240-259 (2007)) is consistent with either mechanism.

Changes in D3 expression and function may signal a sensitive period of development, when drug imprinting is likely to occur (Andersen, Trends Pharmacol. Sci., 26:237-243 (2005)). Drug-induced perturbations before the completion of dopamine receptor development may prevent these developmental rearrangements. The data presented in FIG. 3 suggest that D3 mRNA is overproduced in the MPFCx at P40, but comparable at P20 and P60. MPH prevents this overproduction by reducing D3 mRNA levels by 28.9% when assessed at P40, and this decrease is maintained at P60 (23.8% decrease relative to controls). Mechanistically, brain-derived neurotrophic factor (BDNF) may play a (direct) role in reducing D3 expression as suggested by Le Foll et al. (Eur. J. Neurosci., 15:2016-2026 (2002)) and prevent the overproduction of D3 mRNA. Preliminary results have shown that BDNF mRNA was reduced, and protein levels were modestly, but significantly, lower in MPH-exposed subjects in response to cocaine challenge (data not shown). Because increased BDNF levels have been directly related to sensitization (Le Foll et al., Eur. J. Neurosci., 15:2016-2026 (2002); Hall et al., Neuropsychopharmacology, 28:1485-1490 (2003)), these results raise the possibility that the juvenile drug-exposed MPFCx is less plastic (i.e. less responsive to growth factors).

The majority of research has examined the effects of D3 receptor activity in relation to locomotor activity. D3-preferring agonists, including ±7-OHDPAT and PD 128907, inhibit locomotor activity in both mature (Richtand et al., Neuropsychopharmacology, 28:1422-1432 (2003)) and immature animals (Frantz et al., Eur. J. Pharmacol., 302:1-6 (1996)). Juvenile exposure to both MPH (Andersen et al., 2002) and ±7-OHDPAT (FIG. 6) reduces the locomotor effects of cocaine challenge 25 days post-exposure. This observation is consistent with the reduced locomotor sensitization to amphetamine when the animals were pre-treated with the D3 antagonist Naf (Richtand et al., Brain Res., 867:239-242 (2000)) or reinstatement to cocaine-seeking behavior with SB-277011-A (Cervo et al., Int. J. Neuropsychopharmacol., 10, 167-181 (2007)). However, pre-exposure to a D3 agonist during adulthood did not significantly reduce sensitization, nor did it modulate accumbens D3 receptor density (Richtand et al., Neuropsychopharmacology, 28:1422-1432 (2003)), suggesting that drug imprinting no longer occurs in adulthood (Andersen, Trends Pharmacol. Sci., 26:237-243 (2005)).

Drug-seeking behaviors have typically been associated with D3 changes in the nucleus accumbens, not the MPFCx (Staley et al, J. Neurosci., 16:6100-6106 (1996); Vorel et al., J. Neurosci., 22:9595-9603 (2002); Neisewander et al., Neuropsychopharmacology, 29:1479-1487 (2004); Heidbreder et al., Brain Res. Brain Res. Rev., 49:77-105. (2005); Le Foll et al., Neuropharmacology, 49:525-541 (2005); Gal et al., Drug Alcohol Depend., 81:63-70 (2006)). Here, systemic administration of D3 agonists or antagonists during development does not differentiate the MPFCx or the accumbens for cue-related effects. Our findings that D3 mRNA was reduced in the MPFCx, but not the accumbens or striatum, suggest the MPFCx. We further support a cortical locus of action through the use of ±7-OHDPAT microinjections in the MPFCx. Direct D3 receptor stimulation produced a place preference for cocaine in the MPH-exposed subjects, while having no greater effect in the VEH-exposed subjects. The data suggest that preadolescent exposure to MPH alters the programming of the cortical D3 receptor.

In conclusion, these data demonstrate that exposure to MPH before puberty produces permanent alterations in rCBV that are apparent under MPH challenge in adulthood. These effects are mediated by a reduction in the cortical D3 receptor, and are further associated with a reduction in drug-seeking behavior. Changes in cortical D3 receptors are also associated with novelty seeking (Pritchard et al., Brain Res. Bull., 70:296-303 (2006)), another symptom of ADHD that has been previously linked to D4 receptors (LaHoste et al., Mol. Psychiatry, 1:121-124 (1996)).

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of ameliorating a symptom of a psychiatric disorder in a subject by altering the developmental trajectory of a pre-pubescent brain in a subject predisposed to said disorder comprising the steps of:
(a) providing a pre-pubescent subject predisposed to a psychiatric disorder; and
(b) administering to said subject a psychotropic agent that produces a neurochemical effect characteristic of said disorder in an amount sufficient to imprint on said brain a functional change that ameliorates a symptom of said disorder.

2. The method of claim 1, wherein said disorder is not a substance abuse disorder.

3. The method of claim 1, wherein said disorder is schizophrenia and said psychotropic agent is a dopamine agonist.

4. The method of claim 1, wherein said disorder is depression and said psychotropic agent is a serotonin antagonist.

5. The method of claim 1, wherein said disorder is PTSD and said psychotropic agent is a serotonin antagonist.

6. The method of claim 1, wherein said disorder is Tourette's syndrome and said psychotropic agent is a dopamine agonist.

7. The method of claim 1, wherein said disorder is ADHD and said psychotropic agent is a dopamine agonist.

8. The method of claim 1, wherein said disorder is obsessive compulsive disorder and said psychotropic agent is a serotonin agonist.

9. The method of claim 1, wherein said psychotropic agent is administered at a low dosage.

10. The method of claim 1, wherein said psychotropic agent is administered at a high dosage.

11. The method of claim 1, wherein said psychotropic agent is administered at a moderate dosage.

12. The method of claim 1, wherein said subject is from 6 to 12 years old.

13. The method of claim 1, wherein said psychotropic agent is administered to said pre-pubescent subject for a period of 6 months to 36 months.

14. The method of claim 1, wherein said psychotropic agent is administered to said pre-pubescent subject intermittently.

15. The method of claim 1, further comprising making a determination about whether a pre-pubescent subject is predisposed to a psychiatric disorder on the basis of a genetic test, a cognitive test, a behavioral test, or by taking a family history.

16. A method of ameliorating a symptom of an addiction in a subject by altering the developmental trajectory of a pre-pubescent brain in a subject predisposed to said addiction comprising the steps of:
(a) providing a pre-pubescent subject predisposed to addiction; and
(b) administering to said subject a D3 dopamine agonist in an amount sufficient to imprint on said brain a functional change that ameliorates a symptom of addiction.

17. The method of claim 16, wherein said addiction is a substance abuse disorder.

18. A kit comprising (i) a psychotropic agent; and (ii) instructions for administering said psychotropic agent to a pre-pubescent subject to treat or ameliorate a symptom of a psychiatric disorder, wherein said psychotropic agent produces a neurochemical effect characteristic of said disorder.

19. The kit of claim 18, wherein said disorder is schizophrenia and said psychotropic agent is a dopamine agonist.

20. The kit of claim 18, wherein said disorder is depression and said psychotropic agent is a serotonin antagonist.

21. The kit of claim 18, wherein said disorder is PTSD and said psychotropic agent is a serotonin antagonist.

22. The kit of claim 18, wherein said disorder is Tourette's syndrome and said psychotropic agent is a dopamine agonist.

23. The kit of claim 18, wherein said disorder is ADHD and said psychotropic agent is a dopamine agonist.

24. The kit of claim 18, wherein said disorder is addiction and said psychotropic agent is a D3 dopamine agonist.

25. The kit of claim 18, wherein said disorder is obsessive compulsive disorder and said psychotropic agent is a serotonin agonist.

26. The kit of claim 18, further comprising instructions to administer said psychotropic agent to said pre-pubescent subject for a period of 6 months to 36 months.

27. The kit of claim 18, further comprising instructions to administer said psychotropic agent to said pre-pubescent subject intermittently.
